(19) United States
(12) Patent Application Publication
(10) Pub. No.: US 2010/0239660 A1
(43) Pub. Date: Sep. 23, 2010

(54) PRODUCT AND USE OF OMEGA-3S MATCHING HUMAN TISSUE RATIOS FOR TREATMENT OF INFLAMMATORY AND OTHER CONDITIONS

(76) Inventor: Scott D. Doughman, Chapel Hill, NC (US)

Correspondence Address: PASSE' INTELLECTUAL PROPERTY, LLC, 1717 BRASSFIELD RD., RALEIGH, NC, 27614, US

(21) Appl. No.: 12/727,006

(22) Filed: Mar. 18, 2010

Related U.S. Application Data
(60) Provisional application No. 61/161,647, filed on Mar. 19, 2009.

(51) Int. Cl. A61K 31/202; A61K 9/48; A61P 3/06

(52) U.S. Cl. 424/451; 514/560

(57) ABSTRACT

The present invention relates to a product and method for delivering of DHA and EPA in need of omega-3 treatment. It involves administering a liquid formulation such as an oil containing DHA and EPA in a ratio that closely resembles the ratio of DHA and EPA in the human body. The result is a composition that performs as good or better than fish based compositions containing omega-3 fatty acids and can be dosed into smaller volumes when administering mega doses of 1000-2000 mg of DHA to a selected patient.

This application claims priority of provisional application 61/161,647 filed on Mar. 19, 2009 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the use of vegetarian microalgae omega-3 fatty acids or the equivalent component ratio's of omega-3 fatty acids in the treatment of inflammatory medical conditions. In particular, the present invention relates to the use of omega-3 fatty acids isolated from microalgae in omega-3 ratios close to or matching human organ tissue ratios for DHA and EPA that show improved treatment for lowering postprandial triglycerides and inflammatory conditions and raising HDLs compared to ratio's in fish oils. It is, in some embodiments, useful to treat individuals with glycemic control issues advised away from fish oils and/or animal sources or other sources high in EPA omega-3 content when compared to DHA content.

2. Description of Related Art

Omega-3 fatty acids are a family of unsaturated fatty acids that have a common final carbon-carbon double bond in the n-3 position that is the third bond from the methyl end of the fatty acid. Important nutritionally, essential omega-3 fatty acids are alpha-linolenic acid (ALA), eicosapentaenoic acid (EPA), and docosahesaenoic acid (DHA) all of which are polyunsaturated. The human body cannot synthesize the omega-3 fatty acids de novo but it can form 20- and 22-carbon unsaturated fatty acids from the 18 carbon fatty acid alpha linolenic acid. Both the n-3 and n-6 linoleic acid are essential nutrients which must be obtained from food.

Research has suggested that fish oil, which contains EPA/DHA in a 18/12 ratio (and other ratios isolated from fish oils or artificially produced), reduces risk of ischemic and thrombotic stroke, cancer and other diseases related to the inflammatory process. However, very large amounts of EPA may actually increase the risk of hemorrhagic stroke or further interfere with non-insulin dependent diabetes glycemic controls. However, DHA amounts are not related to these risks and 3 grams of DHA daily is considered safe.

Research has suggested that the in-vitro anti-inflammatory activity of omega-3 fatty acids translates into a clinical benefit. Patients with neck pain and rheumatoid arthritis have demonstrated benefits comparable to treatment with NSAIDs. Those who follow a Mediterranean-style diet tend to have less heart disease, higher HDL cholesterol levels and higher proportions of omega-3 fatty acids in tissues. Other diets including that of the Inuit who also consume large amounts of omega-3 fatty acids from their fatty fish diet have also shown health benefits.

While fish oil capsules and the consumption of omega-3 containing fatty fish has been recommended, the problems with consuming too much fish both from the consumption of mercury and other environmental poisons to the caloric and digestive problems associated with fish in general have added to the problem. In addition, large amounts of the fish oil capsules must be ingested in order to attain desired amounts of these compositions. The American Heart Association recommends that people with high triglycerides take 1000 to 2000 mg each of EPA and DHA. Since most fish oil related capsules only contain about 120 mg of DHA that requires 8 to 20 capsules per day. The ratio of DHA/EPA in most fish oils is only one to two or so, such that most recommendations have been based on the ratios only available in most commercially available fish oil supplements.

Algae oils have DHA/EPA ratios of 24/6 or 30 to zero, entirely different from that in fish oils. Vegetarian microalgae is also a source of EPA and DHA. One particular source is Schizochytrium, from which one can isolate DHA/EPA in a variety of ratios but now can be isolated in a ratio of about 30/1. They are generally equal to the more expensive fish oils, but do not suffer from many of the contamination, odor or reflux problems of fish oil derived DHA. What is not presently known in the art is whether DHA-rich microalgae oil can function as does fish oil for treatment of inflammation or other omega-3 fatty acid sensitive diseases which have entirely different DHA/EPA ratios. The clinical efficacy of DHA rich oil, especially microalgae oil, in the art is unknown (Current Diabetes Reviews, 2007, 3, incorporated herein by reference).

### BRIEF SUMMARY OF THE INVENTION

The present invention relates to the discovery that high DHA based supplements acts as good as or better than standard ratio treatments of DHA/EPA normally from fish oil. In addition, the present invention has higher overall concentrations of DHA which allows delivery in a much smaller number of capsules than fish oil. Particularly useful are ratios of DHA/EPA that are close to body tissue ratios in microalgae omerga-3s such that these produce oils that operate better than fish oil ratios or some regular algae DHA-only products in producing an omega-3 effect in an individual.

In one embodiment, the present invention relates to a method of treating an individual with omega-3 fatty acids comprising administering to an individual in need of omega-3 fatty acid treatment, a composition comprising DHA and EPA in a ratio of from about 25/1 to about 35/1.

In yet another embodiment of the present invention, it relates to a liquid composition for treating a human in need of omega-3 fatty acid treatment comprising DHA and EPA in a ratio of from about 25/1 to about 35/1.

In yet another embodiment, it relates to a method of protecting beta-cells from inflammatory and oxidative stressors comprising daily administering to a human diagnosed as having diabetes a composition comprising DHA and EPA in a ratio of from about 25/1 to about 35/1 wherein the composition comprises 1000 to 2000 mg of DHA.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “treating an individual with omega-3 fatty acids” refers to the administration of a composition comprising at least the two omega-3 fatty acids DHA and EPA. The treatment involves administering an effective dose of a liquid such as an oil containing at least the two omega-3 fatty acids. Treatment is usually oral, with the composition in the form of an oil inside a gel capsule. In one embodiment, the gel capsule is a plant based gel capsule, which has been found to interact less with DHA and EPA than gelatin based capsules derived from an animal source.

Other fatty acids such as ALA can also be included in the formulation herein for treatment. An “omega-3 fatty acid” can mean natural or synthetic omega-3 fatty acids as well as pharmaceutically acceptable esters, derivatives, precursors or salts as well as mixtures thereof. Other omega-3 fatty acids include ALA, esters of omega-3 fatty acids with glycerol, such as mono-, di- and triglycerides, esters of the omega-3 fatty acid and derivatives such as polyglycolized derivatives or polyoxyethylene derivatives. In one embodiment of the present invention the DHA and EPA are isolated from a microalgae such as Schizochytrium.

As used herein an “individual in need of an omega-3 fatty acid treatment” refers to treating an individual with the composition of the present invention in an amount and over sufficient time to treat those disease states which can be treated with omega-3 fatty acids such as coronary heart disease, arrhythmias, cancers, atherosclerosis, neurotransmitter diseases, high triglycerides, aging related issues, inflammation and the like. Many other uses for omega-3 fatty acids are known, for example, as described in U.S. Pat. No. 6,284,268 and incorporated by reference herein. Another use is for treatment of individual with insulin independent type 2 diabetes with composition of the present invention. It is discovered that the composition provides protection of beta-cells from environmental and native inflammatory and oxidative stressors without significant glucose control issues, a concern with high EPA fish oil mixtures.

As used herein the treatment is with a “composition comprising DHA and EPA in a ratio of from about 25/1 to about 35/1” refers to DHA rich microalgae derived DHA/EPA oil or artificially created DHA/EPA oil with such a ratio. It has been discovered that DHA rich compositions of the present invention exhibit as much if not more clinical efficacy than their counterpart fish oil based omega-3 fatty acids oils. The inventor's surmise, though do not wish to be held to a particular theory, that since the ratios of the present invention are closer matched to the ratios of omega-3 fatty acids in the human body, that their efficacy is enhanced when used as a therapeutic agent for administration. The ratio in the human brain of DHA to EPA is approximately 30/1. Therefore one embodiment of the present invention relates to treatment where the ratio is about 30/1.

In one embodiment of the present invention, DHA and EPA are artificially mixed in the ratios of the present invention. For example, DHA and EPA could independently be mixed in a carrier liquid, such as an oil, in the desired ratios. In other embodiments, a naturally occurring product such as a fish or plant based product containing DHA and EPA can have one or the other omega-3 added or subtracted from the formulation in order to attain the proper ratio. In one embodiment, the composition is isolated as is from a natural microalgae such as Schizochytrium which in some species has a naturally occurring ratio of about 30/1. The effects of this ratio have not previously been taught wherein ratios of 13 to 1 were previously known in this species. In the present invention a single dose for daily treatment is where the composition delivers 1000 to 2000 mg of DHA in the daily format. Where it is in an animal derived source it tends to be 8 to 20 capsules but in the plant derived source or when benefiting from mixing the omega-3 fatty acids in from scratch. When using the microalgae derived DHA/EPA one embodiment of treatment is capsules containing 300 mg of DHA and 10 mg of EPA with a 4, 5, 6 or more capsule daily dosage. This is a tremendous advantage for DHA rich treatments over fish oil which requires taking twice as many capsules of the same or larger size. In fact, with the plant based softgel type capsules containing the liquid oil, the plant based capsule allows the capsule dosage to last longer by 50 to 100% without decline such that full potency appears more effective and lasts for a longer period of time than the same microalgae oil placed in animal based gelatin softgel type capsules. Compliance is also better since there is no fish smell or taste with microalgae based formulations. A microalgae or other algae or artificial based formulation becomes yet another embodiment of the present invention.

Since the vegetarian diet largely depends upon ALA to be synthesized into DHA and EPA, it is significant that these fatty acids can be gotten from a vegetarian source. This is especially true because even ALA supplementation is limited. Although one could supplement the present composition with approximately a 1 to 1 ratio with the EPA. In other words, one embodiment of the present invention includes supplementing the plant derived product with sufficient ALA to achieve an DHA/EPA/ALA ratio of about 30/1/1.

A particular advantage of microalgae based compositions such as from Schizochytrium, is that the DHA rich oil obtained from this algae has no unpleasant flavors, no detectable environmental pollutants, and may be supplied in a number of formulations as desired. This microalgae can be rapidly grown in culture where tons of carbon dioxide is removed from the atmosphere for each ton of oil produced making the product more environmentally stable and over production resistant when compared to fish.

The present compositions can have other compositions added to it or coadministered as desired. Vitamins could also be added as desired with the composition of the present invention. One particular co-administered composition includes administering the present composition with a statin type drug. Statins have the undesirable effect of lowering serum omega-3 fatty acids and can act to increase inflammatory actions in the body. Administering the present invention along with a statin, therefore, can alleviate many of the statin based problems associated with their administration.

### EXAMPLE 1

Plant based softgel type gelatin capsules were filled with a microalgae oil having a DHA/EPA ratio of about 30/1. Each capsule was filled such that a capsule contains 300 mg of DHA and 10 mg of EPA. In another example, capsules are similarly filled but 10 mg of ALA are additionally added to each capsule.

### EXAMPLE 2

The capsules of Example 1 are administered 4 a day, to individuals having measured triglycerides in excess of 200 mg/dl in a double blind placebo controlled trial. After administration of the capsules daily for a period of three months the individuals in the trial are re-measured for triglycerides and the individuals measure lowered triglyceride levels in some cases lower than 200 mg/dl. The clinical efficacy has thus been determined to be at least that of fish oil. The compliance with algae oil though is much improved due to the need of taking only 4 capsules instead of 10 fish oil capsules. The improved capsule treatment is particularly advantageous for lowering postprandial triglycerides and inflammatory conditions and raising HDLs over 3 months time. It is in some embodiments useful to treat diabetic individuals with glycemic control issues advised away from fish oils and/or animal sources high in EPA omega-3 content when compared to high DHA content with low EPA present in proportion to the human tissue composition.

The administration of the present invention formulation composition can be made and administered in many forms as one skilled in the art could easily determine in view of the disclosure herein. Variations in the sources of DHA/EPA and the other omega-3 fatty acids is well within the skill in the art in view of the teaching of the clinical efficacy of this particular ratios and of microalgae derived DHA/EPA formulations. According to the claims which follow, these are not intended to be limited by the examples or any particular embodiment of the invention disclosed herein unless so limited by the applicant.

What is claimed is:

1. A method of treating an individual with omega-3 fatty acids comprising administering to an individual in need of omega-3 fatty acid treatment a composition comprising an effective amount of DHA and EPA in a ratio of from about 25/1 to about 35/1.

2. A method according to claim 1 wherein the ratio is about 30/1.

3. A method according to claim 1 wherein the composition is an oil isolated from a microalgae.

4. A method according to claim 3 wherein the microalgae is a Schizochytrium containing DHA and EPA.

5. A method according to claim 1 wherein the composition comprises from about 1000 to about 2000 mg of DHA.

6. A method according to claim 5 wherein the composition comprises about 1200 mg of DHA.

7. A method according to claim 6 wherein the composition is divided into 4 separate dosages of 300 mg DHA each and contained in a softgel capsule.

8. A method according to claim 6 wherein the composition is divided into 3 capsules of 400 mg DHA each.

9. A method according to claim 7 wherein the composition contains at least 35 mg EPA in a 35/1 ratio.

10. A method according to claim 8 wherein the composition contains at least 35 mg EPA in a 35/1 ratio.

11. A method according to claim 1 wherein the composition further comprises ALA in an DHA/EPA/ALA ratio of about 30/1/1.

12. A method according to claim 7 wherein each of the material comprising the softgel capsule is a plant-based formulation.

13. A method according to claim 12 wherein the composition is contained and visible within the capsule as a plant based oil.

14. A method according to claim 13 wherein the oil is a golden microalgae oil.

15. A liquid composition for treating a human in need of omega-3 fatty acid treatment comprising DHA and EPA in a ratio of from about 25/1 to about 35/1.

16. A composition according to claim 15 wherein the liquid is an oil and that both DHA and EPA are soluble in the liquid oil.

17. A composition according to claim 16 wherein the ratio is about 30/1.

18. A composition according to claim 15 wherein the composition is an oil isolated from a microalgae.

19. A composition according to claim 18 wherein the microalgae is a Schizochytrium containing DHA and EPA.

20. A composition according to claim 13 wherein the composition comprises from about 1000 to about 2000 mg of DHA.

21. A composition according to claim 15 wherein the composition comprises about 1200 mg of DHA.

22. A composition according to claim 21 wherein the 1200 mg is divided into 4 separate dosages of 300 mg each contained in a softgel capsule.

23. A composition according to claim 21 wherein the 1200 mg is divided into 3 separate dosages of 400 mg each, wherein each dosage is contained within a softgel capsule.

24. A composition according to claim 21 wherein the composition contains at least 35 mg EPA.

25. A composition according to claim 15 wherein the composition further comprises ALA in a DHA/EPA/ALA ratio of about 30/1/1.

26. A composition according to claim 23 wherein each capsule is a plant softgel based formula.

27. A composition according to claim 15 wherein the composition further comprises a statin drug for lowering cholesterol and triglycerides and raising HDLs.

28. A method according to claim 1 for treating lowering postprandial triglycerides and associated inflammatory conditions and raising HDLs in an individual comprising daily administering a composition comprising about 1200 mg of DHA and about 40 mg of EPA.

29. A method of protecting beta-cells from inflammatory and oxidative stressors comprising daily administering to a human having diagnosed as having diabetes a composition comprising DHA and EPA in a ratio of from about 25/1 to about 35/1 wherein the composition comprises 1000 to 2000 mg of DHA.
